(19) United States
(12) Patent Application Publication
(10) Pub. No.: US 2018/0185575 A1
(43) Pub. Date: Jul. 5, 2018

(54) DEVICE FOR ADMINISTERING FLUID TO A PATIENT

(71) Applicant: B.Braun Melsungen AG, Melsungen (DE)

(72) Inventor: KARSTEN HASLBECK, Melsungen (DE)

(21) Appl. No.: 15/320,647

(22) PCT Filed: Jun. 15, 2015
(86) PCT No.: PCT/EP2015/063289

§ 371 Date: Dec. 20, 2016

(30) Foreign Application Priority Data
Jun. 25, 2014 (DE) 102014212237.6

(51) Int. Cl. A61M 5/168

(52) U.S. Cl. CPC A61M 5/16881; A61M 2005/1405

(57) ABSTRACT

A device for administering fluid to a patient comprising: a fluid source for providing fluid, a patient end which is designed to connect to a patient, a continuous throughflow path connecting the fluid source and the patient end for continuously supplying fluid from the fluid source to the patient end, a bolus flow path connected to the fluid source and to the patient end and arranged in parallel to the continuous throughflow path for supplying the bolus dose of fluid, wherein the bolus flow path is provided with a bolus reservoir for storing the bolus dose and a manually adjustable throughflow contro, adjustable by an operating person, which is designed for manual adjustment of a flow rate, wherein the manually adjustable throughflow control is arranged in the bolus flow path parallel to the bolus reservoir.

### CROSS REFERENCE TO RELATED APPLICATIONS

This application is the U.S. national phase application of PCT International Application No. PCT/EP2015/063289 filed Jun. 15, 2015, which claims priority to German Patent Application No. DE 10 2014 212 237.6 filed Jun. 25, 2014, the contents of each application being incorporated by reference herein.

### FIELD OF THE INVENTION

The invention relates to a device for administering fluid to a patient.

### BACKGROUND OF THE INVENTION

Such devices are of importance in particular in patient controlled analgesia (PCA), The purpose of analgesia is to continuously supply patients with analgesics in order to suppress pain. To alleviate pain attacks exceeding an average level of pain it is known to provide a patient with an actuation element which, when actuated, triggers the administration of an additional dose of analgesics (bolus).

For this purpose a bolus reservoir is provided in which the bolus for the fluid is held. The bolus reservoir may be arranged in a bolus flow path that connects the fluid source with a patient port. A continuous throughflow path connects the fluid source with the patient port for a continuous supply of the fluid from the fluid source to the patient. As soon as the administration of a bolus is triggered, the same is supplied in addition from the bolus reservoir to the patient port via the bolus flow path.

### DESCRIPTION OF THE RELATED ART

EP 1 395 315 B1 and U.S. Pat. No. 6,981,967 B2 describe a device for administering large-volume boli. The manually adjustable throughflow controller is arranged in the continuous throughflow path and is configured such that it controls a flow rate in the continuous throughflow path. It is a drawback of this solution that possible pressure peaks of the fluid delivered may damage the bolus reservoir and/or the bolus flow path.

### SUMMARY OF THE INVENTION

It is an object to provide an improved fluid administration device in which the bolus reservoir is protected against damage caused by overpressure.

The device of the present invention is defined by the features of the independent claim.

According thereto, the manually adjustable throughflow controller is arranged in the adjustable bolus flow path and not in the continuous flow path. Thus, the flow controller does not control the flow rate in the controllable throughflow path, but the flow rate in the bolus flow path. The throughflow controller is arranged in the bolus flow path in parallel to the bolus reservoir. The bolus flow path has two mutually parallel partial paths, one of which includes the bolus reservoir, while the other includes the manually adjustable throughflow controller. That means that the manually adjustable throughflow controller connects the fluid source and the patient end via the bolus flow path.

In case of a pressure peak which may occur e.g. when the fluid source (pump reservoir) is compressed, the flow rate through the continuous throughflow path is increased. In this event, the flow rate through the continuous throughflow path is not limited by the manually adjustable throughflow controller so that the pressure peak can escape via the continuous throughflow path without damaging the bolus reservoir. The manually adjustable throughflow controller is arranged in the bolus flow path and not in the continuous flow path.

Advantageously, the flow rate through the continuous throughflow path is predefined, e.g. by a flow limiter, and is not manually adjustable. In particular, no manually adjustable controller is provided in the continuous throughflow path and the throughflow cannot be varied. The flow rate through the continuous throughflow path may be in the range from 0.1 ml/h (milliliters per hour) to 2 ml/h.

The manually adjustable throughflow controller can adjust the throughflow through the bolus flow path in a range from 0 ml/h and 14 ml/h.

The bolus flow path may have two partial paths, one of the partial parts being associated with the bolus reservoir, while the other partial path is associated with the manually adjustable throughflow controller. The difference between the partial path of the bolus flow path including the manually adjustable throughflow controller and the continuous throughflow path should be seen in the fact that the flow rate through the continuous throughflow path is generally smaller that the flow rate through the partial path including the manually adjustable throughflow controller.

The continuous throughflow path may be provided with a flow throttle. The flow throttle may e.g. be the flow resistance of the continuous throughflow path or a separate component.

The fluid source preferably is a fluid delivery pump pressurizing the fluid to be administered to the patient to a pressure higher than atmospheric pressure.

The patient end may be a port adapted to be connected to a catheter or an injection needle.

The continuous throughflow path and the bolus flow path are advantageously connected with the fluid source via a common inlet path. The continuous throughflow path and the bolus flow path are advantageously connected with the patient end via an outlet path. The continuous throughflow path and the bolus flow path branch from the inlet path and/or open into the outlet path.

A flow indicator may be provided in the inlet path and/or the outlet path, the flow indicator being designed to indicate, whether fluid flows through the corresponding path. In the simplest case the flow indicator may be a transparent tube through which fluid is visible in the corresponding path. As an alternative, it may also be a display indicating the flow rate. A prism is also conceivable as a flow indicator, the reflective/diffractive properties of which change when a fluid flows around the prism.

The continuous throughflow path may include a pressure relief valve which may discharge an overpressure in the system into a collecting reservoir.

Only the flow rate of the bolus flow path is manually adjustable, not the flow rate of the continuous throughflow path.

The bolus reservoir is provided with an actuation element allowing an operator to trigger a bolus delivery by actuation of the actuation element. The actuation element may e.g. be a button that releases a spring-operated piston, wherein fluid is expelled from the bolus reservoir by a displacement of the piston. As an alternative, the bolus reservoir may be an elastically expanding fluid reservoir, e.g. in the form of a balloon, which expands when fluid flows in. The lock is released automatically and the actuation element is enabled for actuation as soon as a predetermined amount of fluid has flown into the bolus reservoir. Such a lock is described in German Patent Application 10 2013 213 529.7 which is incorporated into the present description by reference.

### BRIEF DESCRIPTION OF THE DRAWING

The invention is best understood from the following detailed description when read in connection with the accompanying drawing.

FIG. 1 is a schematic illustration of an embodiment of the invention.

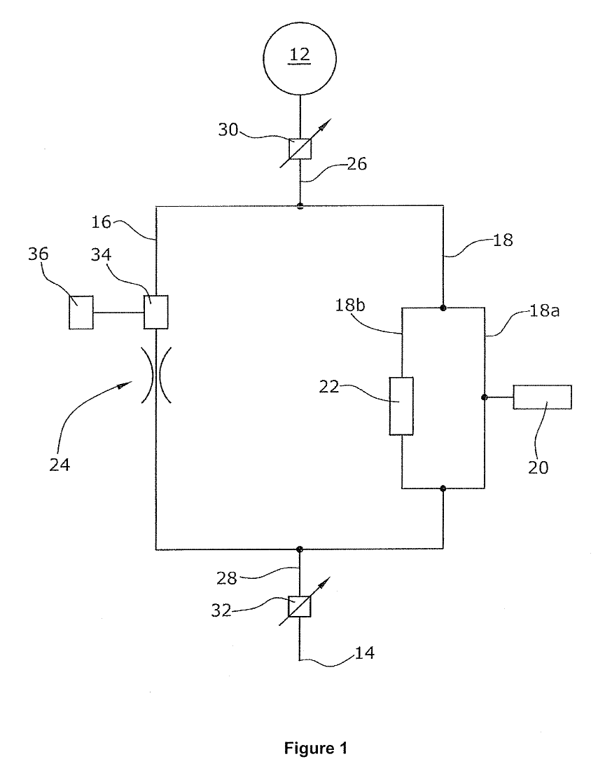

### DETAILED DESCRIPTION OF THE PREFERRED EMBODIMENTS

The fluid source 12 is a fluid delivery pump and is connected in a fluid-conducting manner with the continuous throughflow path 16 and the bolus flow path 18 via an inlet path. The continuous throughflow path 16 and the bolus path 18 branch from the inlet path as separate flow paths. The continuous throughflow path 16 and the bolus flow path 18 are arranged in parallel with each other with respect to the fluid source 12 and the patient end 14.

The continuous throughflow path 16 is provided with a flow throttle 24 that predefines the flow rate of the continuous throughflow path 16. The flow rate through the continuous throughflow path 16 is not variable. The continuous throughflow path includes a pressure relief valve 34 discharging an overpressure in the system into a collecting reservoir 36.

The bolus flow path 18 has two partial paths 18a, 18b arranged in parallel with each other. The bolus reservoir 20 branches from the first partial path 18a. The bolus reservoir 20 is a fluid reservoir for holding a bolus dose of fluid that may be administered to the patient in a pulse-like manner. The fluid typically is an analgesic. It is known in patient controlled analgesia (PCA) that a patient can himself trigger the delivery of a bolus by actuation of a corresponding actuation element (switch). In this regard it is also known that, after a bolus has been triggered, the interval for a delivery of another bolus is predefined. Thereby, overdosing is avoided. For this purpose, a flow limiter is provided in the partial path 18a that defines the time required to fill the bolus reservoir 20 with fluid. The bolus reservoir 20 is designed to be able to trigger a bolus only when the reservoir 20 is filled to a certain degree.

A manually adjustable throughflow controller 22 is provided in the second partial path 18b of the bolus path 18. However, the continuous throughflow path 16 is not provided with a manually adjustable throughflow controller. The manually adjustable throughflow controller 22 is designed to set a follow rate in a range from 0 ml/h and 14 ml/h.

The two partial paths 18a, 18b open into a common line of the bolus flow path 18. The continuous throughflow path 16 and the bolus flow 18 open into a common outlet path 28 that is connected with the patient end 14 in a fluid-conducting manner. A fluid indicator 32 is provided in the outlet path 28. The fluid indicator 32 visually indicates whether fluid flows through the outlet path 28.

1.-12. (canceled)

13. Device for administering fluid to a patient comprising:
a fluid source configured to provide fluid;
a patient end adapted to be connected to a patient;
a continuous throughflow path connecting the fluid source and the patient end for continuously supplying fluid from the fluid source to the patient end;
a bolus flow path connected to the fluid source and to the patient end and arranged in parallel to the continuous throughflow path for supplying a bolus dose of fluid, wherein the bolus flow path is provided with a bolus reservoir for storing the bolus dose; and
a manually adjustable throughflow controller, adjustable by an operator, which is configured for manual adjustment of a flow rate, the manually adjustable throughflow controller arranged in the bolus flow path parallel to the bolus reservoir.

14. Device of claim 13, wherein the manually adjustable throughflow controller is not arranged in the continuous throughflow path.

15. Device of claim 13, wherein the bolus flow path has two parallel partial paths, the bolus reservoir being connected only to the first partial path and the manually adjustable throughflow controller being connected only to the second partial path.

16. Device of claim 13, wherein the fluid source comprises a fluid delivery pump.

17. Device of claim 13, wherein the continuous throughflow path comprises a flow limiter.

18. Device of claim 13, wherein the continuous throughflow path and the bolus flow path are connected with the fluid source via at least one of a common inlet path or the patient end via a common outlet path.

19. Device of the claim 18, wherein at least one of the inlet path or the outlet path comprise a flow indicator to indicate a flow through at least one of the inlet path or the outlet path.

20. Device of claim 13, wherein the flow rate through the continuous throughflow path is predefined and cannot be varied manually.

21. Device of claim 13, wherein the flow rate through the continuous throughflow path is in a range from 0.1 ml/h to 2 ml/h.

22. Device of claim 13, wherein the manually adjustable throughflow controller allows adjustment of the flow rate in a range from 0 ml/h to 14 ml/h.

23. Device of claim 13, wherein the bolus flow path comprises a flow limiter to predefine a time for filling the bolus reservoir.

24. Device of claim 13, wherein the bolus reservoir has an actuation element for triggering a bolus and a lock preventing the actuation of the actuation element, said lock configured to be released after a predefined amount of fluid has flown into the bolus reservoir.
